(19) United States
(12) Patent Application Publication
(10) Pub. No.: US 2016/0287370 A1
(43) Pub. Date: Oct. 6, 2016

(54) BIORESORBABLE, ENDOSCOPIC DCR STENT

(71) Applicant: Daniel A. Rontal, Birmingham, MI (US)

(72) Inventor: Daniel A. Rontal, Birmingham, MI (US)

(21) Appl. No.: 15/085,308

(22) Filed: Mar. 30, 2016

Related U.S. Application Data
(60) Provisional application No. 62/140,170, filed on Mar. 30, 2015.

(51) Int. Cl. A61F 2/04; A61L 31/08; A61L 31/06; A61L 31/16; A61F 2/95; A61L 31/04

(52) U.S. Cl. CPC A61F 2/04; A61F 2/95; A61L 31/041; A61L 31/06; A61L 31/16; A61L 31/08; A61F 2230/0069; A61F 2210/0076; A61L 2300/41; A61L 2420/00; A61L 2300/606; A61L 2300/404

(57) ABSTRACT

A stent and associated method maintains a lacrimal duct in an open, unobstructed state following DCR. The self-expanding, bioresorbable tubular structure has a diameter in the range of 5-8 mm and a length in the range of 4-8 mm following self-expansion. A mesh structure includes struts interconnected through shape-memory hinge regions that open to facilitate expansion. The structure may assume an hour-glass form having a necked-down waist region and flared opposing end regions following expansion to assist in maintaining the stent in position. The stent is compressed to a diameter in the range of 1-3 mm, loaded into an inserter tool, and positioned into a lacrimal duct having an inner wall. The stent is inserted into the lacrimal duct, whereby the stent self-expands into a structure that conformally applies outward pressure to the inner wall of the lacrimal duct to hold open the duct, and the inserter tool is removed.

### REFERENCE TO RELATED APPLICATION

This application claims priority to U.S. Provisional Patent Application Ser. No. 62/140,170, filed Mar. 30, 2015, the entire content of which is incorporated herein by reference.

### FIELD OF THE INVENTION

This invention relates generally to dacryocystorhinostomy, or DCR, and, more particularly, to a bioresorbable stent to keep the neo-ostium open, and decrease granulation and scarring after the surgery.

### BACKGROUND OF THE INVENTION

Epiphora, or excessive tearing, can result from several different etiologies but blocked lacrimal ducts is one of the most common reasons. This blockage is frequently fixed with a surgical procedure called dacryocystorhinostomy, or DCR, which opens the lacrimal system into the nose. At the end of the procedure, tubes are commonly placed through the puntae of the eye, passed into the nose, and tied internally into the nose.

Currently there is debate as to whether the tubes benefit the procedure, or worsen the results by causing granulation tissue and subsequent scarring. The currently used Crawford tubes are kept in place for months before removal. They are uncomfortable to patients, as they completely plug the outflow of the tears causing epiphora (the reason the procedure was performed in the first place), since no flow is allow through the system while the tubes are in place. Those of skill in this art will appreciate that alternative devices and methods would improve the post-op DCR procedures.

### SUMMARY OF THE INVENTION

This invention improves upon DCR procedures post-op by providing a bioresorbable stent to keep the neo-ostium open while decreasing granulation and scarring after the surgery. As no tubes or other structures would be left in the punctae, post-operative epiphora should also be greatly reduced.

A stent according to the invention for maintaining a lacrimal duct in an open, unobstructed condition following a dacryocystorhinostomy (DCR) procedure comprises a self-expanding, bioresorbable tubular structure having a diameter in the range of 5-8 mm and a length in the range of 4-8 mm following self-expansion. In the preferred embodiment, structure is a mesh comprising struts interconnected through shape-memory hinge regions that open to facilitate expansion. To achieve bioresorbability, the stent is preferably composed of a mixture of poly lactic acid (PLA) and poly lactic-co-glycolic acid (PLGA) or poly glycolic acid (PGA) and PGLA. The structure may include a steroid and/or Mitomycin-C.

In a preferred embodiment, the structure assumes an hour-glass form having a necked-down waist region and flared opposing end regions following expansion to assist in maintaining the stent in position. The structure may have a diameter in the range of 1-3 mm when compressed for insertion.

A method of maintaining a lacrimal duct in an open, unobstructed condition following a dacryocystorhinostomy (DCR) procedure, comprises the step providing a self-expanding, bioresorbable stent having a diameter in the range of 5-8 mm and a length in the range of 4-8mm following self-expansion. The stent is compressed, loaded into an inserter tool, and the tool is positioned into a lacrimal duct having an inner wall. The stent is inserted into the lacrimal duct, whereby the stent self-expands into a structure that conformally applies outward pressure to the inner wall of the lacrimal duct to hold open the duct; and the inserter tool is removed from the operative site.

### BRIEF DESCRIPTION OF THE DRAWINGS

FIG. 1 is a drawing that illustrates a self-expanding, bioresorbable stent according to the invention;

FIG. 2 shows the stent of claim 1 compressed and loaded into an inserter instrument; and

FIG. 3 is a simplified illustration that shows a deployed stent assuming an hour-glass profile.

### DETAILED DESCRIPTION OF THE INVENTION

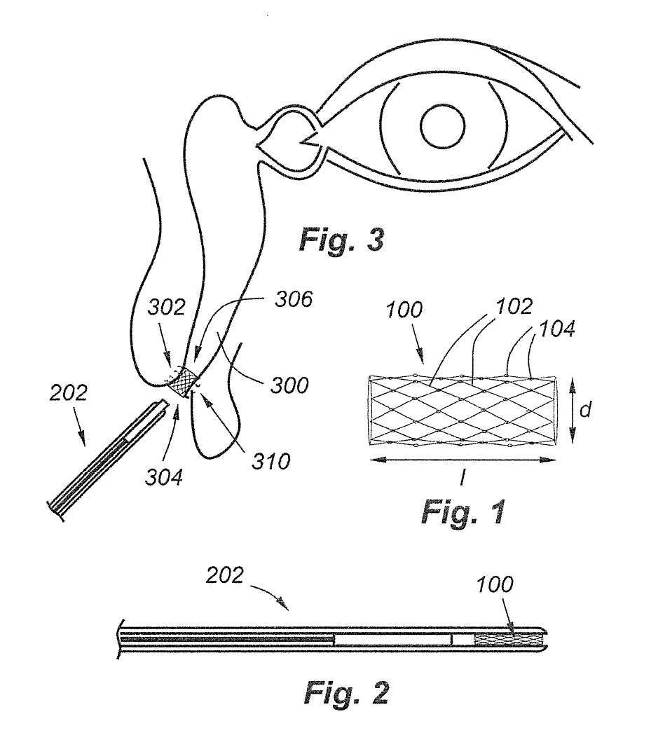

In broad and general terms, this invention resides in a self-expanding, bioresorbable stent used to hold open the lacrimal duct opening after dacryocystorhinostomy (DCR). As such, as shown in FIG. 1, the stent would be in the form of a self-expanding tube-like structure 100 measuring 5-8 mm in diameter (d) and 4-8 mm in length (l) following self-expansion.

To achieve bioresorbability, the stent is composed of a mixture of poly lactic acid (PLA) and poly lactic-co-glycolic acid (PLGA) or poly glycolic acid (PGA) and PGLA, with structural integrity lasting a variable length of time depending on the exact utility required. Typically this would be in the range of 3-6 weeks. While the structure may be solid, in the preferred embodiments the structure is a mesh comprising struts 102 interconnected through hinge regions 104. The hinges connecting each strut provide the spring action operative to expand the stent following placement.

The structure of the stent enables the device to conform to the shape of the opening created in the lacrimal system by the surgeon. When fully expanded, the stent would hold the shape of a circular ring in cross section, but due to the malleable nature of the materials it could hold the shape as an oval, ellipse, circle or irregular shape depending upon the anatomy of the lacrimal duct.

In terms of the operative procedure, the stent is compressed and loaded into an introducer apparatus to then be placed into the neo-ostium. When compressed, the diameter of the device is roughly 2 mm, while the length may increase by several millimeters. FIG. 2 shows the stent of claim 1 compressed and loaded into an inserter instrument 202. FIG. 3 is a simplified illustration that shows a deployed stent inserted into the neo-ostium of the lacrimal duct 300. As shown, the preferred embodiment the structure includes a waist region 302 in the middle of the device, with the ends 304, 306, expanding wider than the waist. Between this “hour-glass” shape and the radial spring force, the stent would stay in place in the opening of the duct. Small prongs 310, protruding from the intra-luminal portion of the device, may be provided to assist in holding the stent in position.

DCR surgery is limited by granulation and scarring. Studies range from 2.6% to 20.7% as to the incidence of granulation after the procedure, leading to scarring and revision surgery. Thus, while one important function of the stent is to physically hold the surgically created ostium open following the procedure, in the preferred embodiment the device is steroid-coated to apply a high steroid dose directly to the site of surgical trauma while avoiding the deleterious effects of systemic steroids. A highly hydrophobic steroid would reduce the systemic absorbtion as well as reduce the absorbtion across the tissues into the orbit and globe. Analagous stents in other parts of the nose have shown the ability to reduce scarring by up to 70%. Alternatively, other compounds could be used to coat the stent, such as Mitomycin-C.

1. A stent for maintaining a lacrimal duct in an open, unobstructed condition following a dacryocystorhinostomy (DCR) procedure, comprising:
a self-expanding, tubular structure composed of a bioresorbable material; and
wherein the structure measures 5-8 mm in diameter and 4-8 mm in length following self-expansion.

2. The stent of claim 1, wherein the structure is a mesh comprising struts interconnected through hinge regions.

3. The stent of claim 1, wherein the stent is composed of a mixture of poly lactic acid (PLA) and poly lactic-co-glycolic acid (PLGA) or poly glycolic acid (PGA) and PGLA.

4. The stent of claim 1, wherein the structure includes a highly hydrophobic steroid.

5. The stent of claim 1, wherein the structure is coated with Mitomycin-C.

6. The stent of claim 1 wherein, following self-expansion, the structure assumes an hour-glass form having a necked-down waist region and flared opposing end regions.

7. The stent of claim 1, wherein the structure has a diameter in the range of 1-3 mm when compressed for insertion.

8. A method of maintaining a lacrimal duct in an open, unobstructed condition following a dacryocystorhinostomy (DCR) procedure, comprising the steps of:
providing the stent of claim 1;
compressing the stent, and loading the stent into an inserter tool;
positioning the inserter tool into a lacrimal duct having an inner wall;
inserting the stent into the lacrimal duct, whereby the stent self-expands into a structure that conformally applies outward pressure to the inner wall of the lacrimal duct to hold open the duct; and
removing the inserter tool.

9. The method of claim 8, wherein the stent has a diameter in the range of 1-3 mm when compressed for insertion.

10. The method of claim 8, wherein the stent expands into a structure measuring 5-8 mm in diameter and 4-8 mm in length.

11. The method of claim 8, wherein the stent expands into a structure having an hour-glass form with a necked-down waist region and flared opposing end regions.

12. The method of claim 8, wherein the stent is composed of a mixture of poly lactic acid (PLA) and poly lactic-co-glycolic acid (PLGA) or poly glycolic acid (PGA) and PGLA.

13. The method of claim 1, wherein the stent delivers a highly hydrophobic steroid.

14. The method of claim 1, including a coating of Mitomycin-C.
